(19) United States
(12) Patent Application Publication
(10) Pub. No.: US 2020/0239857 A1
(43) Pub. Date: Jul. 30, 2020

(54) STRETCHABLE SKIN-ON-A-CHIP

(71) Applicants: INDUSTRY ACADAMIC COOPERATION FOUNDATION, HALLYM UNIVERSITY, Chuncheon-si, Gangwon-do (KR); RESEARCH & BUSINESS FOUNDATION SUNGKYUNKWAN UNIVERSISTY, Suwon-si, Gyeonggi-do (KR)

(72) Inventors: Gun Yong SUNG, Seoul (KR); Ho Yeong LIM, Anseong-si (KR); Hyun Jeong SONG, Anseong-si (KR); Sungsu PARK, Seoul (KR)

(21) Appl. No.: 16/478,446

(22) PCT Filed: Dec. 29, 2017
(86) PCT No.: PCT/KR2017/015720

§ 371 Date: Jul. 16, 2019

(30) Foreign Application Priority Data
Jan. 16, 2017 (KR) 10-2017-0007160
Dec. 27, 2017 (KR) 10-2017-0180679

(51) Int. Cl. C12N 5/071; C12M 3/00; C12M 3/06; C12M 1/00; G01N 33/50

(52) U.S. Cl. CPC C12N 5/0698; C12M 21/08; C12M 23/16; C12N 2533/40; G01N 33/5044; C12N 2513/00; C12M 23/22

(57) ABSTRACT

Disclosed is a skin-on-a-chip, which more closely resembles real skin by simulating the repetition of contraction and relaxation due to stretching of skin cells, by embedding a permanent magnet in the skin-on-a-chip. The skin-on-a-chip includes a connector that causes a linear motion in the skin cells of the chip when driven by a linear drive device outside the chip, which provides forward and backward movement, to thereby simulate contraction and relaxation of skin.

### TECHNICAL FIELD

The present invention relates to a skin-on-a-chip, and more particularly to a skin-on-a-chip, which includes therein a connector that causes linear motion in the skin cells of the chip when driven by an external linear motion drive device, and is thus capable of culturing artificial skin more closely resembling real skin by simulating repeated contraction and relaxation due to stretching of skin cells.

The present invention was supported by the Basic Research Laboratory Program (Grant ID: NRF-2015R1A4A1041631) for “3D Printing-based Human-on-Chip Fusion Lab” of the Ministry of Science and ICT, Republic of Korea.

### BACKGROUND ART

An organ-on-a-chip is a technique for culturing cells that constitute a certain living organ on a chip having electronic circuits to thereby mimic mechanical and physiological cellular responses as well as the functions and characteristics of the corresponding organ. It enables detailed study of the mechanism of physicochemical reaction or cell movement of a certain organ and is also expected to be useful as a model for new drug development or toxicity assessment.

The roles of electronic circuits vary depending on the kind of organ-on-a-chip, but are commonly responsible for mimicking the microenvironment inside the organ and sensing the physiological response to thus display the same as data. A lung-on-a-chip mimics the flow of air and body fluid inside the body with a culture medium system using electronic circuits, and in an eye-on-a-chip, electronic circuits are used to periodically supply tears through compartmentalization depending on the kinds of cells.

The first organ-on-a-chip was a lung-on-a-chip, in which lung cells are cultured together with electronic circuits on a small plastic chip of about 3 cm, developed by Dongeun Huh, a professor of bioengineering at the University of Pennsylvania in 2010, at the Wyss Institute, USA. In a method of manufacturing a microchip, a micro cell culture environment is constructed on a chip in which electronic circuits are installed, and living lung cells and vascular cells are cultured in a perfusion chamber, so as to exchange oxygen and carbon dioxide through pulmonary alveoli and capillaries resembling real lungs. This lung-on-a-chip is able to simulate the progress of lung-related disease after infection therewith and to observe the same in real time. Moreover, it is known to be capable of simulating complications caused by side effects of chemotherapy, rather than a single disease.

After development of the lung-on-a-chip, the organ-on-a-chip was manufactured for a variety of organ models such as the heart, eyes, arteries, kidneys, skin, and the like, and is used to study the mechanism of physicochemical reaction or cell movement of the organ in detail, and is also expected to be useful as a model for new drug development or toxicity assessment (see Wikipedia).

Since the human skin is exposed to various chemicals and biological agents such as cosmetics, detergents, ultraviolet rays, pathogens, environmental pollutants and microorganisms, the skin plays a main role in protecting organs by providing a physiological barrier. Increased levels of these chemicals and biological agents on the skin may cause diverse reactions such as skin inflammation, itching, allergies, and even tumors. Hence, it is necessary to filter the toxicity of such external substances and also to enhance the effects of drugs used for the skin. To this end, millions of animals, including mice, have been employed worldwide for experiments. However, animal testing has two crucial limitations. The first is an ethical problem, and second, there are significant differences between mouse skin and human skin in terms of thickness, hair density and appendages. Furthermore, mice have no sweat glands on the skin except on the soles of the feet. As reported by the International Humane Society, nine out of ten drug candidates that are found to be safe and effective upon animal testing fail when subsequently tested on humans, and animal testing often fails to predict the results of real application to humans. For this reason, there has been a need to establish an alternative in-vitro system that mimics human skin as closely as possible. Since the first report of human-skin-like structures in the early 1980s, various in-vitro skin models have been developed and commercialized. However, most of these models employ a static culture system that mimics only the human epithelium based on fibroblasts and keratinocytes. The complicated structure of the skin cannot be mimicked using these cells alone. This is because the skin contains pores, immune cells, melanocytes, Merkel cell complexes, blood vessels, nerve fibers and multilayer structures. Thus, many researchers in industrial, clinical and academic fields participate in developing in-vitro skin models that may mimic skin and skin diseases.

Most 3D-based cell cultures that have been studied to date have been performed in a static environment [Y. G. Anissimov et al., Advanced drug delivery reviews, 65, 169-190 (2013)]. However, stimuli are being continually applied inside and outside the human body. On the skin of the human body, external contact and friction, muscle relaxation and contraction, and the like are always occurring during daily life. The currently reported studies include increased stratum corneum differentiation in a stretching environment [Nobuhito Mori et al., IEEE MEMS 24-28 Jan. 2016], inhibition of the expression of olfactory ensheathing cells [Kamble Harshad et al., Biomed microdevice. 19. May (2016)], etc. Therefore, it is impossible to observe skin changes due to skin relaxation and contraction by a conventional static skin-on-a-chip.

### DISCLOSURE

### Technical Problem

Therefore, the present invention has been made keeping in mind the problems encountered in a conventional static skin-on-a-chip and is intended to provide a skin-on-a-chip that mimics a state more similar to human skin conditions, that is, the state in which contraction and relaxation occur repeatedly.

### Technical Solution

In the present invention, a skin-on-a-chip, which includes therein a connector that causes linear motion in the skin cells of the chip when driven by an external linear motion drive device in order to simulate external stimuli applied to the skin, was manufactured, and cell culture behavior was observed by applying a mechanical stimulus to the chip intermittently or periodically during cell culture using the external drive device. In the present invention, the skin-on-a-chip was designed so as to correspond to a contraction of about 10% by determining the contraction rate caused by the change in elasticity depending on the proportion of polydimethylsiloxane (PDMS) (i.e. a mixing ratio of PDMS and a curing agent), and was standardized using an aluminum mold. Furthermore, a plate that enables fixation to a plate of a square dish size so as to apply a constant stimulus all of the time was designed.

Experiments adopted a manner of applying a 10% contraction stimulus at a frequency of 0.01 Hz at 12 hr/day in consideration of sleep and rest times of the human body during daily life. The tissue cultured in a conventional static environment and the tissue obtained after stretching were subjected to H/E staining and compared and analyzed using an optical microscope.

Based on the results of observation of the cross-sections of the tissues through H/E staining when stretching was applied to the skin-on-a-chip and when a static state was maintained, differentiation of fibroblasts and keratinocytes showed a great difference. When stretching was performed on a skin-on-a-chip having a permanent magnet embedded therein, keratinocytes were confirmed to grow in the state of being attached well to collagen segments even after the lapse of time, and also, when stretching was applied, keratinocytes were confirmed to gradually infiltrate a collagen fibroblast layer due to stress over time. Accordingly, the skin-on-a-chip, which three-dimensionally cultures skin cells, is expected to be useful in testing cytotoxicity, etc. by making skin cells more closely resemble real skin through intermittent stretching.

As used herein, the term “skin equivalent” refers to skin cells cultured on a stretchable skin-on-a-chip of the present invention.

Shape of Fibroblasts and Changes in Expression Levels of ECM protein and β-actin

The present inventors measured changes in the expression level and shape of fibroblasts by observing the tissues in samples using pig skin collagen and rat tail collagen, and quantitatively measured the change in expression level of β-actin through gene analysis. β-actin is a protein that is essential for living cells and is important to the cytoskeleton. This is generally used as a control as a housekeeping gene in experiments, but it is not suitable for use as a control when cell aging occurs. Thus, such changes in cytoskeleton may be regarded as evidence of aging in skin equivalents stimulated by stretching. Moreover, H/E tissue-staining results showed that long fibroblasts observed in samples under static conditions were aged due to stretching and thus took on a round, small elliptical shape.

Fibroblasts play a major role in the production of protein responsible for the extracellular matrix (ECM), for example, collagen, fibronectin, etc. However, the extracellular matrix expression capacity is decreased in aged fibroblasts, resulting in the formation of fragile skin and wrinkled skin. In the present invention, the extracellular matrix expression capacity was confirmed to significantly decrease in skin equivalent samples after 7 days of stretching. In the experimental group using pig skin collagen, the number of fibroblasts decreased as the stimulation cycle was shorter, and thus if a stretching stimulus is applied at a fast cycle (5.3 mm/s, 0.05 Hz), an environment in which it is difficult for cells to live is created, resulting in cell death, and the stimulation also affects the expression of collagen and fibronectin. In the experiment using rat tail collagen, it was confirmed that the fibroblast size was significantly changed in the experimental group subjected to stretching. Under static conditions shown in FIG. 6(d), the fibroblasts had a long shape, and under stretching conditions shown in FIG. 6(h), the fibroblasts had a round elliptical shape. As such, the length of the fibroblasts was about 49.8±12 μm under static conditions and was 11.8±6.8 μm under stretching conditions, in which the difference therebetween was about five times. This suggests that the stretching stimulus causes a large change in the cytoskeleton itself. With regard thereto, based on the results of quantitative analysis of β-actin, it can be seen that the length gradually decreases as stretching is applied [FIG. 10(a)]. Immunohistochemical staining results also revealed significant differences in the production of fibronectin and collagen [FIG. 9]. Under stretching conditions, the newly formed collagen cannot extend into a fiber shape but is formed as a fragile skeleton having a round ring shape. Accordingly, it can be found that the stretching conditions affect the cytoskeleton of fibroblasts and thus the expression of extracellular matrix protein is decreased, and also that fragile skin resembling aged skin is formed, and therefore the aging of fibroblasts is promoted by the physical stimulus.

Aging of Stratum Corneum Under Stretching Conditions

In the present invention, when a fast stimulus was applied to a skin equivalent using a stretching device (5.3 mm/s), the stratum corneum also showed a great difference from the culture in a static environment. Based on the results of H/E staining, as stimulation was accelerated in skin equivalents using pig skin collagen, the stratum corneum became thinner and more fragile and was thus stripped from the dermal layer, and in the sample on the 7th day of stimulation, keratinocytes penetrated into the dermal layer and thus wrinkled skin was formed [FIG. 4]. Also, keratin 10 expression was decreased in the case of 0.01 Hz stimulation for 7 days compared to the static environment, and in particular, the expression level was remarkably decreased at 0.05 Hz. Based on the above results, the support was formed with rat tail collagen (0.85 wt %), which is regarded as more suitable for 3D cell culture, and treated in the static environment and the 0.01 Hz stretching environment. Based on the results of H/E staining, the thickness of the stratum corneum was 86.4±26 μm under static conditions and was 49.8±12 μm under stretching conditions and thus the stratum corneum was experimentally confirmed to be thinned by about 37 μm upon stretching, and on the 7th day of stretching, the keratinocytes were observed to penetrate into the dermis due to stress [FIG. 6]. This is considered to be because the cells migrate to a safe place due to stress applied thereto, and in this procedure, the skin is wrinkled. Also, as in the above experiment, the expression level of keratin 10 was remarkably lowered under stretching conditions, which means that the expression level of keratinocytes is decreased.

In order to prove the phenomena by which the stratum corneum is weakly attached and the wrinkled skin is formed under stretching conditions, filaggrin, laminin α5, and involucrin, important proteins in the stratum corneum, were quantified and compared using qPCR. With regard to filaggrin, which is responsible for protecting and moisturizing the skin, it was found that the expression thereof was remarkably decreased from the day of stretching and then gradually increased to the normal level, indicating that the expression level, decreased by stretching, affected the rate of keratinization and the expression occurred to a level similar to the static environment on the 7th day, at which the stratum corneum was fully formed. Laminin α5, which plays an important supporting role in the stratum basale, contributes to reduction in expression and formation of wrinkles upon aging, and the expression level of laminin α5 upon stretching for 1 day and 3 days was similar to that of the static environment, but the expression level of laminin α5 was decreased on the 7th day, indicative of the beginning of aging in the cells. The results of tissue staining show that, on the 7th day, keratinocytes penetrated into the dermal layer and the wrinkled skin was formed and thus cell aging began. Involucrin is responsible for skin protection, and the expression level thereof significantly decreases when stretching is applied, and then continues to decrease as stretching is continually applied. This phenomenon explains the fact that the stratum corneum gradually becomes thinner and more fragile.

Increased Expression of P53 in Aged Skin

The expression of P53, which is a gene that repairs mutant cells or cancer cells or induces apoptosis, tends to increase in aged skin. Therefore, the skin was confirmed to be aged based on an increase in P53 upon stimulation of skin equivalents using a stretching device. For 1 day and 3 days, the P53 level was lower than the normal level, which is deemed to be because cell activity was reduced due to stress. However, on the 7th day of stimulation, the P53 level increased 2.5 times compared to the 3^{rd} day and was higher than the normal level. This indicates that the expression of P53 was increased due to the formation of mutant cells or cancer cells, from which the expression level thereof may be regarded as greatly increased taking into consideration the reduction in the cell activity due to stress. Therefore, an increase in the P53 level, which is a typical phenomenon in aged cells, was confirmed on the 7th day, and thus it can be found that skin aging is caused by mechanical stimulation through the stretching device.

Aged Skin Equivalent

Skin aging was observed in stretching-stimulated samples, and samples subjected to stretching stimulus for 3 days, 5 days and 7 days were compared to check the date on which aged skin equivalents were formed. On the 3^{rd} day of stretching, ECM expression tended to decrease in skin equivalents, but the stratum corneum was not fully formed, the expression of laminin α5 was not decreased, and the expression of P53 was not increased. Also, on the 5th day of stretching stimulation, the keratinocytes did not penetrate into the dermal layer and wrinkled skin was not formed. However, on the 7th day of stretching stimulation, the keratinocytes penetrated into the fragile stratum corneum and the dermal layer, and also, the formation of wrinkled skin, decreased expression of ECM protein, and increased P53 were confirmed, indicating that skin aging, rather than instantaneous stress, was caused on the 7th day of stretching. Thus, it is deemed that skin aging was promoted upon stimulation for 7 days under fast stimulus conditions (5.3 mm/s) [FIG. 11].

The stratum corneum appeared to be thicker in skin equivalents cultured with vascular cells in the Takeuchi group, in which skin equivalents were studied using a stretching device. However, when stimuli were applied to skin equivalents using the stretching device manufactured in the present invention, different results were obtained. The reason why the opposite results are obtained under stretching conditions is that, in the Takeuchi group, cell activity was affected because of slow modification using clamps, thus activating cell proliferation. In contrast, the stretching device manufactured in the present invention is deemed to cause a great difference in the stress in the cells because the stretching stimulus was applied at a very high speed (5.3 mm/s), and thus the high-speed stretching acts as a stress on the cells, whereby a difference in activity occurs, ultimately promoting aging of the cells with regard to the shape and function thereof. It has been experimentally confirmed that if stretching stimulus is continually applied for 7 days or more, the formation of the wrinkled skin and the degradation of the protein expression capacity to protect and support the skin occur, and thereby a skin equivalent resembling aged skin is formed.

In order to mimic the skin of an aged person, in the present invention, a stretching device that operates at a high speed of 5.3 mm/s using a permanent magnet and an electromagnet was manufactured, whereby the skin equivalent was contracted and relaxed at a frequency of 0.01 Hz. Phenomena occurring in aged skin, such as wrinkled skin, fragile stratum corneum, reduction in the expression level of protein related to moisturization and support, and increased expression of P53, upon stretching of 3D skin equivalents made of human skin cells, are described below.

First, changes in the dermal layer were caused by stimulation through a stretching device, in which the long shape of fibroblasts was changed to a round shape having a length of about ⅕ of an original length thereof, and thus the expression of extracellular matrix proteins, for example, collagen and fibronectin, was remarkably decreased, and qPCR analysis showed that the expression level of β-actin, which is important to the cytoskeleton, gradually decreased under conditions such that the stimulus was continually applied. These results appear to indicate that fibroblast expression is decreased by stretching stimulation.

Second, the stratum corneum thickness, keratinocyte behavior and protein expression decreased in the stratum corneum. The thickness of the stratum corneum tended to decrease by about ½ of that in the static environment, and the keratinocytes penetrated into the dermal layer on the 7th day of stimulation using a stretching device, indicative of the formation of wrinkled skin as in the laminin α5, which tends to decrease on the 7th day. The expression of keratin 10 showed a tendency to decrease with an increase in the stimulus intensity, and there was a great difference in expression between the static environment and the stimulation environment at a frequency of 0.01 Hz. Also, based on the results of qPCR, the expression level of filaggrin, which is involved in skin protection and moisturization, decreased and then gradually increased, which is deemed to contribute to the delay of the formation of the stratum corneum, and involucrin was continuously decreased in expression level upon stimulation, and a fragile and thin stratum corneum was formed by stretching stimulation.

Third, there is expression of P53, which is an important aging marker that repairs mutant cells or cancer cells, induces apoptosis, and increases expression upon aging. The activity of the dermis and stratum corneum was very low due to stress under stretching conditions, and thus the expression level of P53 on the 1st day and the 3^{rd} day was about ½ compared to the static environment. However, on the 7th day, the expression level thereof was increased about 2.5 times compared to the 3^{rd} day. These results show that the expression of p53 was greatly increased in consideration of the fact that the cells were lowered in activity, indicating that skin aging occurred on the 7th day of stretching stimulation, like the results of keratinocytes penetrating into the fragile stratum corneum and the dermal layer using a stretching device.

Through experiments, when the skin equivalent was stretched by about 10% at a frequency of 0.01 Hz under conditions of 25 V and a permanent-magnet-to-electromagnet distance of 6 mm at a speed of 5.3 mm/s for 7 days, the phenomena occurring in aged skin were confirmed. The stretchable skin-on-a-chip according to the present invention enables testing in a small chip before in-vivo experiments in cosmetics development, drug testing, and the like, and is expected to be useful as a powerful testing tool in place of in-vivo experiments through further studies.

### Advantageous Effects

When stretching is performed on a skin-on-a-chip having a permanent magnet embedded therein according to the present invention, it can be confirmed that keratinocytes grow in the state of being attached well to collagen segments even after the lapse of time, and also that keratinocytes gradually infiltrate a collagen fibroblast layer due to stress over time upon stretching.

Moreover, according to the present invention, compared to skin cells growing under static culture conditions, the skin-on-a-chip of the present invention is capable of simulating actual aged skin through stretching and is thus more closely similar to real skin, making it suitable for use in testing cosmetics, dermatological drugs, toxic substances and the like.

### BRIEF DESCRIPTION OF DRAWINGS

FIG. 1 schematically shows a stretchable skin-on-a-chip, in which the upper layer is configured to include a culture medium chamber, a culture chamber and a permanent magnet, and the lower layer includes a microfluidic channel for supplying a culture medium to the culture chamber for culturing skin cells, and a membrane for preventing the skin cells from being immersed in the culture medium and supplying the culture medium from below the culture chamber;

FIG. 2 shows (a) a graph showing the results of strain depending on the PDMS base:curing agent mixing ratio and the permanent-magnet-to-electromagnet distance at 30 V, and (b) a graph showing the results of strain depending on the PDMS base:curing agent mixing ratio and the permanent-magnet-to-electromagnet distance at 25 V, *- actual conditions used in the experiment (PDMS base:curing agent=35:1, 25 V, about 10% strain);

FIG. 3 shows images of H/E (hematoxylin and eosin) staining of skin equivalents using pig skin collagen, in which (a), (b) and (c) are samples cultured through air exposure for 3 days, 5 days and 7 days under static conditions, (d), (e) and (f) are samples cultured through air exposure for 3 days, 5 days and 7 days under cyclic stretching conditions of 12 hr/day and 0.01 Hz at 25 V, 1 A, and 10% strain, and (g), (h) and (i) are samples cultured through air exposure for 3 days, 5 days and 7 days under cyclic stretching conditions of 12 hr/day and 0.05 Hz at 25 V, 1 A, and 10% strain;

FIG. 4 shows images of special staining of skin equivalents using pig skin collagen, in which (a), (b) and (c) are samples cultured through air exposure for 3 days under static conditions, (d), (e) and (f) are samples cultured through air exposure for 3 days under cyclic stretching conditions of 12 hr/day and 0.01 Hz at 25 V, 1 A, and 10% strain, and (g), (h) and (i) are samples cultured through air exposure for 7 days under cyclic stretching conditions of 12 hr/day and 0.05 Hz at 25 V, 1 A, and 10% strain, (a), (d) and (g)—H/E staining, (b), (e) and (h)— Masson's trichrome staining, (c), (f) and (i)—Sirius staining (optical microscope, 200× magnifications);

FIG. 5 shows immunohistochemical results of skin equivalents using pig skin collagen, in which (a), (b) and (c) are samples cultured through air exposure for 3 days under static conditions, (d), (e) and (f) are samples cultured through air exposure for 3 days under cyclic stretching conditions of 12 hr/day and 0.01 Hz at 25 V, 1 A, and 10% strain, and (g), (h) and (i) are samples cultured through air exposure for 7 days under cyclic stretching conditions of 12 hr/day and 0.05 Hz at 25 V, 1 A, and 10% strain, (a), (d) and (g)—fibronectin staining, (b), (e) and (h)—collagen IV staining, and (c), (f) and (i)—keratin 10 staining (optical microscope, 200× magnifications);

FIG. 6 shows the results of H/E staining analysis of skin equivalents using rat tail collagen, in which (a), (b) and (c) are samples cultured through air exposure for 3 days, 5 days and 7 days under static conditions (optical microscope, 200× magnifications), (d) shows the enlarged fibroblast shape of the box of (b) (optical microscope, 800× magnification), (e), (f) and (g) are samples cultured through air exposure for 3 days, 5 days and 7 days under cyclic stretching conditions of 12 hr/day and 0.01 Hz at 25 V, 1 A, and 10% strain (optical microscope, 200× magnifications), and (h) shows the enlarged fibroblast shape of the box of (f) (optical microscope, 800× magnification);

FIG. 7 shows the results of special staining analysis of skin equivalents using rat tail collagen, in which (a), (b) and (c) are samples cultured through air exposure for 7 days under static conditions, and (d), (e) and (f) are samples cultured through air exposure for 7 days under cyclic stretching conditions of 12 hr/day and 0.01 Hz at 25 V, 1 A, and 10% strain, (a) and (d)—H/E staining images, (b) and (e)—Masson's trichrome staining images, and (c) and (f)—Sirius staining images (optical microscope, 200× magnifications);

FIG. 8 shows the results of immunohistochemical staining analysis of skin equivalents using rat tail collagen, in which (a), (b) and (c) are samples cultured through air exposure for 7 days under static conditions, and (d), (e) and (f) are samples cultured through air exposure for 7 days under cyclic stretching conditions of 12 hr/day and 0.01 Hz at 25 V, 1 A, and 10% strain, (a) and (d)—fibroblast staining images, (b) and (e)—collagen IV staining images, and (c) and (f)—keratin 10 staining images (optical microscope, 200× magnifications);

FIG. 9 shows the results of comparison of skin equivalents under cyclic stretching conditions of 12 hr/day and 0.01 Hz at 25 V, 1 A, and 10% strain, (a), (d) and (g) illustrating the results of H/E staining of samples subjected to stretching for 3 days, 5 days and 7 days, (b), (e) and (h) illustrating the results of fibronectin staining of samples subjected to stretching for 3 days, 5 days and 7 days, and (c), (f) and (i) illustrating the results of collagen IV staining of samples subjected to stretching for 3 days, 5 days and 7 days (optical microscope, 200× magnifications);

FIG. 10 is graphs showing the results of qPCR quantitative analysis, (a) illustrating the expression of β-actin over time in static culture and in the stretchable skin-on-a-chip at 0.01 Hz and 10% strain (air exposure for 0 day, 1 day, 3 days, 7 days), (b) illustrating the expression of filaggrin over time in static culture and in the stretchable skin-on-a-chip at 0.01 Hz and 10% strain (air exposure for 0 day, 1 day, 3 days, 7 days), (c) illustrating the expression of laminin α5 over time in static culture and in the stretchable skin-on-a-chip at 0.01 Hz and 10% strain (air exposure for 0 day, 1 day, 3 days, 7 days), (d) illustrating the expression of involucrin over time in static culture and in the stretchable skin-on-a-chip at 0.01 Hz and 10% strain (air exposure for 0 day, 1 day, 3 days, 7 days), and (e) illustrating the expression of P53 over time in static culture and in the stretchable skin-on-a-chip at 0.01 Hz and 10% strain (air exposure for 0 day, 1 day, 3 days, 7 days); and

FIG. 11 shows changes in skin aging-related protein and gene factors for 3 days and 7 days under stretching conditions.

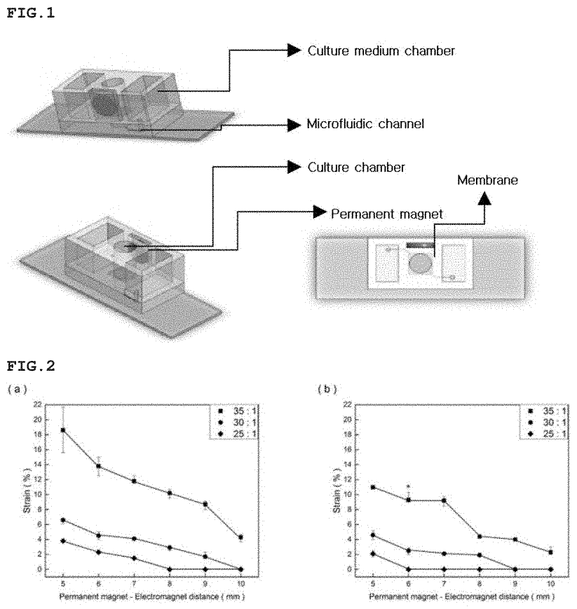

### BEST MODE

The present invention provides a skin-on-a-chip, suitable for use in culturing skin cells by supplying a culture medium to skin cells three-dimensionally arranged on a chip, the skin-on-a-chip including therein a connector that causes linear motion in the skin cells of the chip when driven by a linear drive device outside the chip, which provides linear forward and backward movement, so that the skin cells are stretched to thereby simulate contraction and relaxation of the skin.

Also, in the present invention, the connector may be mechanically, electrically or magnetically connected to the linear drive device outside the chip.

In addition, the present invention provides a skin-on-a-chip, comprising:

a base layer;

a lower layer disposed on the base layer and configured to include a microfluidic channel and a membrane; and

an upper layer disposed on the lower layer and configured to include a culture medium chamber, a skin cell culture chamber for three-dimensionally culturing skin cells, and a connector that may be connected to a linear motion drive device outside the chip, which provides linear forward and backward movement. Here, the connector may be mechanically, electrically or magnetically connected to the linear motion drive device outside the chip.

Furthermore, in the present invention, the linear drive device and the connector may be connected in a variety of connection manners, including a mechanical connection manner between connecting rings, a manner of passing a connecting ring through a through-hole, and the like, in addition to the use of a magnet, a magnetic field or a magnetic object, and the connection manner is not particularly limited so long as it does not interfere with application of a linear motion to the skin cells to cause contraction and relaxation.

Also, in the skin-on-a-chip of the present invention, the base layer may be made of a material comprising or consisting of glass or a transparent synthetic polymer. The base layer may be manufactured using a material such as glass and/or an optically clear synthetic polymer such as polystyrol, polycarbonate, polysiloxane, polydimethylsiloxane, etc.

Also, the microfluidic channel of the lower layer may connect the culture medium chamber and the skin cell culture chamber of the upper layer to supply a culture medium to the skin cells.

Also, in the skin-on-a-chip of the present invention, the membrane of the lower layer may be positioned below the skin cell culture chamber of the upper layer.

Also, in the skin-on-a-chip of the present invention, the connector may be positioned around the skin cell culture chamber.

Also, in the skin-on-a-chip of the present invention, at least one connector may be provided.

Also, in the skin-on-a-chip of the present invention, at least one of the lower layer and the upper layer may be formed of PDMS (polydimethylsiloxane) or a composition including PDMS.

Also, in the skin-on-a-chip of the present invention, the skin cells may comprise at least one of fibroblasts and keratinocytes.

Also, in the skin-on-a-chip of the present invention, the skin cells may be added with a support for three-dimensional cell culture.

Also, in the skin-on-a-chip of the present invention, the support may be at least one biocompatible support selected from the group consisting of collagen, gelatin, fucoidan, alginate, chitosan, hyaluronic acid, silk, polyimide, polyamic acid, polycaprolactone, polyetherimide, nylon, polyaramid, polyvinyl alcohol, polyvinyl pyrrolidone, polybenzyl glutamate, polyphenylene terephthalamide, polyaniline, polyacrylonitrile, polyethylene oxide, polystyrene, cellulose, polyacrylate, polymethyl methacrylate, polylactic acid (PLA), polyglycolic acid (PGA), polylactic acid-co-polyglycolic acid (PLGA), poly{poly(ethylene oxide) terephthalate-co-butylene terephthalate} (PEOT/PBT), polyphosphoester (PPE), polyphosphazene (PPA), polyanhydride (PA), poly(ortho-ester) (POE), poly(propylene fumarate)diacrylate (PPF-DA), and poly(ethylene glycol)diacrylate (PEG-DA).

Also, in the skin-on-a-chip of the present invention, the skin cells may include endothelial cells, dermal cells and epithelial cells.

In addition, the present invention provides a method of evaluating the efficacy of a dermatological composition by intermittently applying a one-way linear motion by a linear drive device outside the skin-on-a-chip described above to cause relaxation and contraction in skin cells so as to simulate skin cells.

Also, the dermatological composition may be a cosmetic composition, a skin external preparation composition or a toxicity test substance.

### MODE FOR INVENTION

A better understanding of the present invention will be given through the following examples, which are merely set forth to illustrate but are not to be construed as limiting the scope of the present invention, as will be apparent to those skilled in the art.

Cell Culture

Human fibroblasts were cultured using a DMEM culture medium (containing 10% (v/v) fetal bovine serum and 1% penicillin/streptomycin), and in the experiment, human fibroblasts were mixed with pig skin type 1 collagen (SK Bioland) or type 1 collagen sol extracted from rat tails, cured for 1 hr in a CO₂ incubator, and then cultured for 4 days while the culture medium was replaced every day. The concentration of the fibroblasts was 2.0×10⁴ cells/ml.

Human keratinocytes were subcultured using a KGM (Lonza) culture medium, and for stratum corneum formation, human keratinocytes (Biosolution Co., Ltd.) were sprayed onto the surface of collagen gel on which fibroblasts were cultured for 4 days and attached for 1 hr in a CO₂ incubator, after which a KGM culture medium was supplied thereto. As such, the concentration of human keratinocytes was 6×10⁶ cells/ml, and culture was performed for 4 days while the culture medium was replaced every day.

A DMEM culture medium was used for culturing the collagen gel to which human fibroblasts were added, and when fibroblasts and keratinocytes were cultured together, DMEM was supplied to the lower layer along the microfluidic channel and KGM was supplied onto the collagen gel of the culture chamber.

In order to induce differentiation of keratinocytes through air exposure, culture was carried out using DMEM/Ham's F12 (10 ng/ml of EGF-1, 0.4 μg/ml of hydrocortisone, 5 μg/ml of insulin, 5 μg/ml of transferrin, 2×10⁻¹¹ M 3,3,5-triiodol-thyonine sodium salt, 10⁻¹⁰ M cholera toxin, 10% (v/v) fetal bovine serum, and 1% penicillin/streptomycin).

Manufacture of Stretchable Skin-On-a-Chip

In the present invention, the culture medium was supplied through the microfluidic channel, whereby human-like skin tissue was cultured three-dimensionally. Furthermore, a structure in which a permanent magnet was inserted into a chip was designed in order to realize a stretchable skin-on-a-chip that provides a physical stimulus without disturbing the supply of culture medium. To this end, the skin-on-a-chip was manufactured so as to include upper and lower layers.

In order to manufacture the upper layer, taking into consideration the culture space and the position of a permanent magnet, a PDMS (polydimethylsiloxane) base and a curing agent were mixed at a ratio of 35:1, placed in an aluminum mold (CSI Tech), and cured in an oven at 80° C. for 1 hr, after which the mold was removed therefrom. Then, a permanent magnet was inserted thereto, and the PDMS mixed solution was poured again and cured in an oven at 80° C. for 1 hr. Thereafter, the lower layer was manufactured in a manner in which a PDMS base and a curing agent were mixed at a ratio of 10:1, poured onto a master pattern wafer patterned with a channel having a width of 150 μm and a height of 50 μm through photolithography, and then cured in an oven at 80° C. for 1 hr, thereby obtaining a lower layer having a fine pattern. The process of adhering the base layer, the lower layer and the upper layer was performed using O₂ plasma (FEMTO Science). The inserted permanent magnet was a disc-shaped neodymium magnet having a diameter of 10 mm and a thickness of 1 mm (JL magnet). As an electromagnet, a circular electromagnet having a diameter of 40 mm (JL magnet) was used, and a magnetic-plate oxide-film-treated aluminum mold (CSI Tech) was used.

Stretching Test

In the stretching test, 10% contraction-relaxation was repeated for 12 hr at a frequency of 0.01 Hz using an alternating voltage of 1 A and 25 V applied to the electromagnet, and the static state was maintained for 12 hr (FIG. 2). This device was driven through PC control, which is advantageous in making convenient and precise measurement.

Histology, IHC Staining, Special Staining

Skin equivalents were fixed in 4% paraformaldehyde and embedded in paraffin. After rehydration, tissue segments (5 mm) were subjected to H/E (hematoxylin and eosin) staining for tissue experiments or immunohistochemistry for certain protein expression studies.

As primary antibodies for fibronectin, cytokeratin 10, CD34 and collagen IV, ab2413 (abcam), ab6318 (abcam), ab81289 (abcam) and ab6586 (abcam) were used, and as a secondary antibody, a rabbit-specific HRP/DAB (ABC) detection kit (ab64261, abcam) was used.

For special staining, MT (Massan Trichrome stain kit Procedure, K7228, IMEB INC) and Sirius red/Fast Green staining were used, and fluorescent slides were visualized and recorded using an OLYMPUS IX173.

qPCR Quantitative Analysis

For qPCR analysis, mRNA was extracted in a manner in which the sample was treated with 1 ml of a triazole reagent and thus RNA was separated from cells and extracted through RNA precipitation, RNA washing, and RNA resuspension, and mRNA was quantified using a Nanodrop 2000C (Thermo). Thereafter, cDNA was synthesized using an amfiRivert cDNA Synthesis Platinum Master Mix (genDEPOT). The purified cDNA was subjected to qPCR quantification using an AccuPower 2X GreenStar™ qPCR Master Mix (Bioneer) through an Exicycler™ 96 (Bioneer). Each primer is given in Table 1 below. The sequences of Table 1 show SEQ ID NOS: 1 to 12.

Result 1: Strain Comparison of Stretchable Skin-On-a-Chip

In order to control the chamber strain of the stretchable skin-on-a-chip, the volume ratio upon PDMS preparation, the distance between the permanent magnet and the electromagnet, and the voltage that was applied to the electromagnet were varied. The PDMS base:curing agent mixing ratio was set to 25:1, 30:1 and 35:1, the distance between the permanent magnet and the electromagnet was set to 5, 6, 8 and 10 mm, and the voltage applied to the electromagnet was set to 25 V and 30 V (FIG. 2).

As shown in FIG. 2, the strain was relatively high, to the level of about 10%, under conditions such that the voltage applied to the electromagnet was 25 V, the PDMS base:curing agent mixing ratio was 35:1 and the permanent-magnet-to-electromagnet distance was 5 mm, and there was little change in strain despite the decreased distance at a ratio of 25:1. The greatest strain was exhibited at a PDMS base:curing agent ratio of 35:1 at 30 V, and about 11% stain appeared even at a distance of 8 mm. In addition, the strain was significantly increased at 30 V compared to 25 V at ratios of 30:1 and 25:1. In order to adopt about 10% strain, a stretchable skin-on-a-chip manufactured at a PDMS base:curing agent ratio of 35:1 at 25 V, in which the permanent magnet and the electromagnet were spaced apart from each other at a distance of 6 mm, was used for experiments.

Result 2: Tissue Analysis of Skin Equivalents Using Pig Skin Collagen Under Static and Stretching Conditions

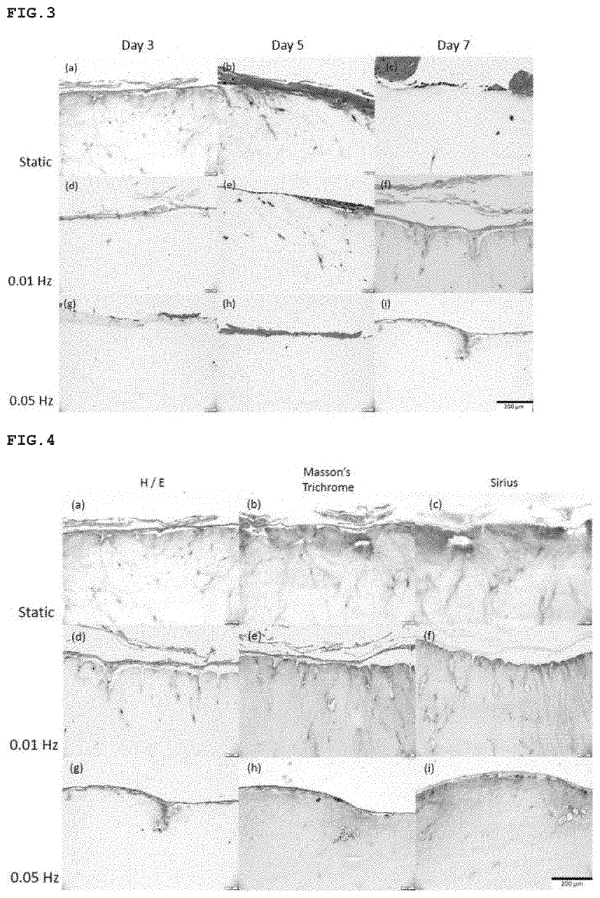

In a comparative group subjected to stretching, culture was carried out through air exposure for 3 days, 5 days and 7 days under cyclic stretching conditions of 12 hr/day and 0.01 Hz and 0.05 Hz at 25 V, 1 A, and 10% strain, and static culture was conducted through air exposure for 3 days, 5 days and 7 days without stretching. The tissue cross-sections were fixed in paraffin and analyzed through H/E staining (FIG. 3).

As shown in FIG. 3, based on the results of observation of the cross-sections of tissues after H/E staining of the samples cultured under stretching and static conditions, fibroblasts and keratinocytes were found to have different results under individual conditions. Under cyclic stretching of 0.01 Hz and 0.05 Hz, the phenomena by which keratinocytes gradually infiltrated the collagen gel due to stress on the 7th day were similarly shown, and when the stimulus was more frequently applied by shortening the stretching cycle, the number of fibroblasts was decreased and a fragile stratum corneum was formed.

Result 3: Masson's Trichrome and Sirius Staining of Skin Equivalents Using Pig Skin Collagen Cultured Under Static and Stretching Conditions

Fibroblasts function to produce extracellular matrix such as collagen, fibronectin, etc. Thus, fibroblasts play an important role in realizing skin elasticity. In order to evaluate whether fibroblasts function properly under stimulation, total collagen production was analyzed through special staining. As special staining, Masson's trichrome staining and Sirius staining were performed. Here, when these two staining results were consistent with each other, the evidence of total collagen production is regarded as valid.

In the Masson's trichrome staining method, the portion stained in dark blue represents the cell nucleus and the stratum corneum is stained in pink. Also, collagen is stained in light blue. In the Sirius staining method, the portion stained in dark pink represents the cell nucleus, the stratum corneum is stained in blue, and the collagen is stained in pink. As shown in FIG. 4, the Masson's trichrome and Sirius staining results in the samples were consistent, collagen expression was high around the epithelial layer under static conditions, collagen expression was generally high in the dermal layer at 0.01 Hz stimulation, and the expression level was generally low at 0.05 Hz stimulation (FIG. 4).

Result 4: Immunohistochemical Staining of Skin Equivalents Using Pig Skin Collagen Under Static and Stretching Conditions

In the dermal layer of the human body, skin produces collagen, various proteins are expressed, and moisture protection and elasticity are increased, and in the stratum corneum, cells die to thus be keratinized, which protects the human body from fungi, bacteria and foreign substances entering the body and prevents moisture loss. For this reason, collagen IV and fibronectin 10 were measured in order to evaluate the collagen production and fibronectin production of fibroblasts in the present invention, and keratin 10 was measured in order to evaluate the normal function of keratinocytes.

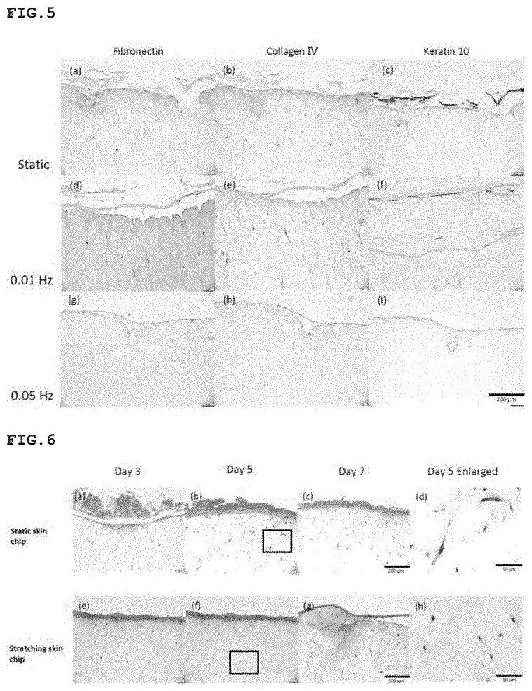

As shown in FIG. 5, in the case of fibronectin and collagen IV, the portion stained in dark brown represents the cell nucleus and the light brown portion is the expression portion of fibronectin and collagen IV. For reference, the portion stained in blue is the stratum corneum. The results of static culture showed that expression occurred in the periphery of the epithelial layer, like the Masson's trichrome and Sirius staining results. However, in the samples cultured at 0.01 Hz stretching, slight expression occurred in the periphery of the epithelial layer. In addition, at 0.05 Hz, fibronectin and collagen IV showed very low expression in a round ring shape, rather than a thread shape. In keratin 10, the keratin in the stratum corneum was stained in dark brown and the cells were stained in blue. Keratin 10 was well expressed in the outer portion, which was exposed to air, in the sample cultured in the static environment. However, in the samples cultured in the stretching environment, slight expression occurred at the top of the collagen layer at 0.01 Hz and expression hardly occurred at 0.05 Hz (FIG. 5).

Result 5: Tissue Analysis of Skin Equivalents Using Rat Tail Collagen Under Static and Stretching Conditions

The above experimental results demonstrated that the skin equivalents using rat tail collagen were more suitable for cells, rather than the skin equivalents using pig skin collagen. Therefore, skin equivalents using 0.85 wt % rat tail collagen were used to compare cell changes when a stretching stimulus was applied to skin equivalents using rat tail collagen, which is more suitable for 3D cell culture.

For tissue analysis, the experiment was carried out under the same culture conditions as in the pig skin collagen results, and using as a support rat tail collagen (0.85 wt %), the samples cultured on the skin-on-a-chip under static conditions and the stretchable skin-on-a-chip under stretching conditions of 0.01 Hz and 10% strain were compared through H/E staining. FIGS. 6(a), (b) and (c) show the cross-sections of the skin equivalents cultured for 3 days, 5 days and 7 days under static conditions, and FIG. 6(d) shows the enlarged image of the rectangular portion of FIG. 6(b). FIGS. 6(e), (f) and (g) show the cross-sections of the skin equivalents cultured for 3 days, 5 days and 7 days under stretching conditions, and FIG. 6(h) shows the enlarged image of the rectangular portion of FIG. 6(f). As for culture through air exposure on the 5th day, the stratum corneum was thicker under static conditions than under stretching conditions, and was confirmed to be well attached to the dermal layer. When compared numerically, the stratum corneum was formed at a thickness of 49.8±12 μm under stretching conditions, which is about 37 μm thinner than 86.4±26 μm under static conditions. Interestingly, a great change in the shape of fibroblasts was observed. Upon each kind of culture through air exposure on the 5th day, the fibroblasts of the skin-on-a-chip under static conditions were extended in a long star-like shape to thus have a length of about 50±24 μm, and the fibroblasts of the stretchable skin-on-a-chip had a round, small elliptical shape with a length of 11.8±6.8 μm.

Result 6: Masson's Trichrome and Sirius Staining of Skin Equivalents Using Rat Tail Collagen Cultured Under Static and Stretching Conditions

The samples on the 7th day of air exposure cultured under static conditions and under stretching conditions were subjected to Masson's trichrome staining and Sirius staining and compared.

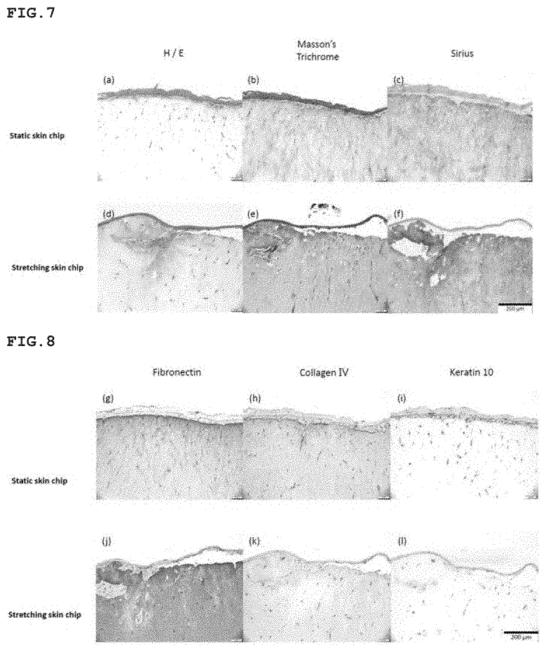

As shown in FIG. 7, the results of Masson's trichrome staining and Sirius staining showed the same tendency under the corresponding conditions. Under static conditions, a lot of collagen newly expressed in thread form appeared in the entire dermal layer and the stratum corneum was more darkly stained. However, under stretching conditions, unlike static conditions, the expression level of collagen in thread form was low, and it was difficult to distinguish it from the rat tail collagen used as the conventional support except for the darkly stained portion around the cells. Thereby, it can be concluded that the collagen expression capacity of fibroblasts was significantly decreased upon application of stress.

Result 7: Immunostaining of Skin Equivalents Using Rat Tail Collagen Cultured Under Static and Stretching Conditions

In order to compare the results of immunostaining, the samples on the 7th day of air exposure used in the above tissue-staining results were also used, and in order to evaluate the normal functions of the two cells, fibronectin expression and collagen IV expression were compared as representative protein synthesis indicators of fibroblasts. To evaluate the protein expression of keratinocytes, keratin 10 expression was compared.

FIGS. 8(g), (h) and (i) are images showing the expression of fibronectin, collagen IV, and keratin 10 of the samples cultured under static conditions. In the entire dermal layer, fibronectin and collagen IV appeared to be well expressed in thread form, which is consistent with the results of special staining. It was also confirmed that keratin 10 was well expressed near the stratum corneum on the epidermal cells. Also, FIGS. 8(j), (k) and (1) are images showing the expression of fibronectin, collagen IV, and keratin 10 of the samples cultured under stretching conditions. As such, fibronectin or collagen IV was not expressed in thread form, as in the tissue cultured under static conditions, and appeared in a round ring shape like the use of pig skin collagen, and was stained much less compared to the case of static conditions. In particular, collagen IV was found to be almost absent in the stretching environment, indicating that it was hardly expressed in the results of Masson's trichrome and Sirius staining and was expressed by the existing rat tail collagen. Moreover, the expression level of keratin 10 was confirmed to be very low when compared with the static conditions.

Result 8: Comparison of Tissue Images of Skin Equivalents Using Rat Tail Collagen Depending on Stretching Time

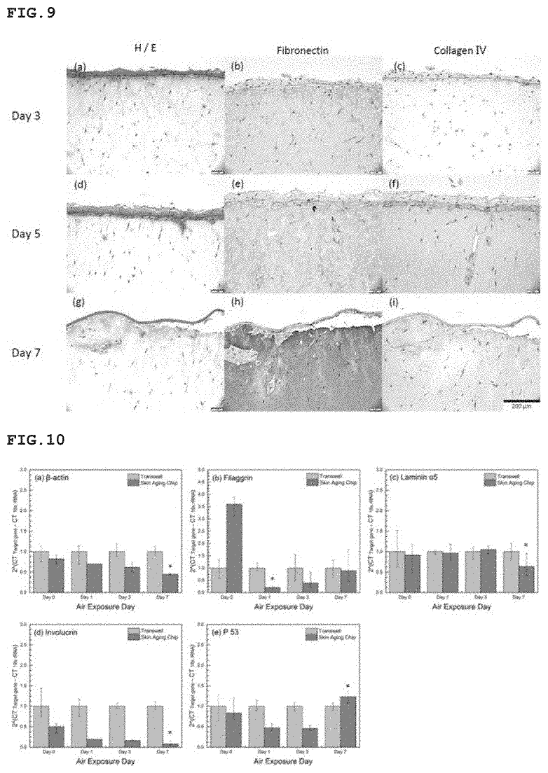

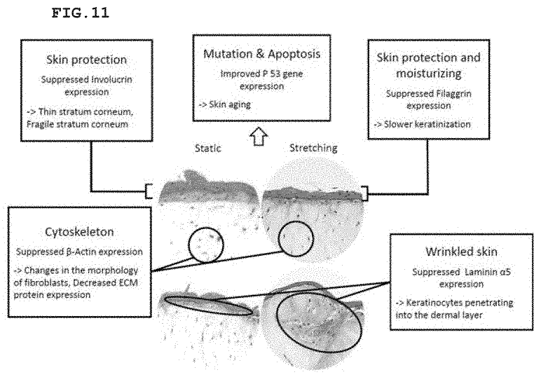

The phenomena by which the stratum corneum was thinned and the number of cells decreased over time were confirmed through tissue images. Thus, in order to evaluate changes in protein expression over time, fibronectin and collagen IV of the samples cultured for 3 days, 5 days, and 7 days were compared through H/E staining and immunohistochemical staining. FIGS. 9(a), (b) and (c) show tissue images of the samples cultured for 3 days under stretching conditions, FIGS. 9(d), (e) and (f) show tissue images of the samples cultured for 5 days under stretching conditions, and FIGS. 9(g), (h) and (i) show tissue images of the samples cultured for 7 days under stretching conditions. It was confirmed that the expression of fibronectin appeared as a round ring shape under stretching conditions, and low expression in the 3^{rd} day samples, higher expression in the 5th day samples, and low expression in the 7th day samples were observed. Moreover, the penetration of keratinocytes on the 7th day of stretching was confirmed through immunohistochemical staining.

Result 9: Analysis of Protein Expression Genes Derived from Skin Equivalents Using Rat Tail Collagen Cultured Under Static and Stretching Conditions

In order to quantitatively analyze the aging phenomena observed through tissue analysis, aging-related factors were compared through qPCR. Among them, filaggrin is a protein that is expressed mainly in keratinocytes and is involved in skin protection and moisturization, and tends to decrease upon aging. Laminin α5 is a protein expressed mainly in the basement membrane at the dermis-stratum corneum junction, plays a role in supporting the skin, and decreases upon aging, thereby causing wrinkled skin. Involucrin is involved in skin protection of the stratum corneum and tends to decrease in aged skin [32], and P53 is a gene that repairs mutant cells and induces apoptosis of aged cells or cancer cells, and the expression level of the P53 gene tends to increase in the aged skin. Furthermore, β-actin is important to the cytoskeleton and is used as a control in typical experiments, but β-actin cannot be used as a suitable control under conditions that cause aging, because there has been reported a decrease in β-actin expression upon aging. As shown in FIG. 10(a), β-actin expression gradually decreased over time, and in FIGS. 10(b) and (c), the expression of filaggrin decreased drastically at the beginning of stretching and then gradually increased, and the expression of laminin α5 was similar to the static conditions but decreased on the 7^{th} day. As shown in FIG. 10(d), the expression of involucrin decreased significantly at the beginning of stretching and gradually decreased over time, and as shown in FIG. 10(e), the expression of P53 gradually decreased on the 1st day and the 3^{rd} day of stretching, and then remarkably increased about 2.5 times on the 7th day compared to the 3^{rd} day of stretching.

TABLE 1

| | Forward | Reverse |
|---|---|---|
| 18s RNA | 5′-GGCGCCCCCTCGAT | 5′-GCTCGGGCCTGCTTT |
| | GCTCTTAG-3′ | GAACACTCT-3′ |
| β-actin | 5′-TTGCTGATCCACAT | 5′-GGCACCCAGCACAAT |
| | CTGCTGGAAG-3′ | GAAGATCAA-3′ |
| Filaggrin | 5′-GGAGTCACGTGGCA | 5′-GGTGTCTAAACCCGG |
| | GTCCTCACA-3′ | ATTCACC-3′ |
| Involucrin | 5′-CCGCAAATGAAACA | 5′-GGATTCCTCATGCTG |
| | GCCAACTCC-3′ | TTCCCAG-3′ |
| Laminin α5 | 5′-GGAACTTCCGGCAT | 5′-GGACAGGCACAGCTC |
| | ACGGAGA-3′ | CACATT-3′ |
| P53 | 5′-CCGCCCAACAACAC | 5′-GGCCTGGGCATCCTT |
| | CAGCTCCT-3′ | GAGTTCC-3′ |

### INDUSTRIAL APPLICABILITY

According to the present invention, a stretchable skin-on-a-chip is useful for testing cosmetics, dermatological drugs, and toxic substances because it can simulate a skin state similar to that of living bodies.

### Sequence Listing Free Text

The sequences of the present invention are primers for performing qPCR on aging-related factors.

1. (canceled)

2. (canceled)

3. A skin-on-a-chip, comprising:
a base layer;
a lower layer disposed on the base layer and configured to include a microfluidic channel and a membrane; and
an upper layer disposed on the lower layer and configured to include a culture medium chamber, a skin cell culture chamber for three-dimensionally culturing skin cells, and a connector that causes a linear motion in the skin cells of the chip when driven by a linear drive device outside the chip, which provides linear forward and backward movement,
wherein the skin cell culture chamber contains a support for three-dimensional culture of skin cells,
wherein the connector is at one side of the skin cell culture chamber,
wherein the membrane is positioned below the skin cell culture chamber in the upper layer, preventing the skin cells from being the skin cells from being immersed in the culture medium,
wherein the microfluidic channel is supplying the culture medium and oxygen to the skin cells and recovering waste materials and carbon dioxide from the skin cells by connection the membrane and the culture medium chamber.

4. The skin-on-a-chip of claim 3, wherein the connector is mechanically, electrically or magnetically connected to the linear drive device outside the chip.

5. (canceled)

6. The skin-on-a-chip of claim 3, wherein the base layer is made of a material comprising or consisting of glass or a transparent synthetic polymer.

7. (canceled)

8. (canceled)

9. (canceled)

10. (canceled)

11. The skin-on-a-chip of claim 3, wherein at least one of the lower layer and the upper layer is formed of PDMS (polydimethylsiloxane) or a composition including PDMS.

12. (canceled)

13. (canceled)

14. The skin-on-a-chip of claim 3, wherein the support is at least one selected from the group consisting of collagen, gelatin, fucoidan, alginate, chitosan, hyaluronic acid, silk, polyimide, polyamic acid, polycaprolactone, polyetherimide, nylon, polyaramid, polyvinyl alcohol, polyvinyl pyrrolidone, polybenzyl glutamate, polyphenylene terephthalamide, polyaniline, polyacrylonitrile, polyethylene oxide, polystyrene, cellulose, polyacrylate, polymethyl methacrylate, polylactic acid (PLA), polyglycolic acid (PGA), polylactic acid-co-polyglycolic acid (PLGA), poly{poly(ethylene oxide)terephthalate-co-butylene terephthalate} (PEOT/PBT), polyphosphoester (PPE), polyphosphazene (PPA), polyanhydride (PA), poly(ortho-ester) (POE), poly(propylene fumarate)diacrylate (PPF-DA), and poly(ethylene glycol)diacrylate (PEG-DA).

15. (canceled)

16. A method of evaluating efficacy of a dermatological composition by intermittently applying a one-way linear motion by a linear drive device outside the skin-on-a-chip of claim 3 to cause relaxation and contraction in skin cells so as to simulate skin cells.

17. The method of claim 16, wherein the dermatological composition is a cosmetic composition, a skin external preparation composition or a toxicity test substance.
